(19) United States
(12) Patent Application Publication
(10) Pub. No.: US 2003/0157159 A1
(43) Pub. Date: Aug. 21, 2003

(54) Prevention and treatment of digestive tract infections

(76) Inventors: Lanny Udell Franklin, Atlanta, GA (US); Julio L. Pimentel, Buford, GA (US)

Correspondence Address: JULIO L. PIMENTEL, Ph.D., 3206 WINDGATE DR., BUFORD, GA, 30519-1941, US

(21) Appl. No.: 10/045,352

(22) Filed: Jan. 15, 2002

(51) Int. Cl. A61K 9/127; A61K 31/05; A61K 31/11; A61K 31/015

(52) U.S. Cl. 424/450; 514/733; 514/693; 514/763

(57) ABSTRACT

Prevention and treatment of digestive tract infections in humans and animals by orally administering a single terpene, a terpene mixture or a liposome-terpene(s) composition before or after the onset of a gastro-intestinal infection. Such infections may include traveler's diarrhea, ulcers, anthrax and other bacterial and parasitical infections.

### BACKGROUND OF THE INVENTION

1. Field of the Invention

Prevention and treatment of digestive tract infections in humans and animals by orally administering a single terpene, a terpene mixture or a liposome-terpene(s) composition before or after the onset of the infection, single terpenes having biocidal activity which in combination with two or more other terpenes synergistically increase the biocidal effectiveness.

2. Discussion of the Background

Digestive tract infections are mainly caused by pathogenic and opportunistic microorganisms and toxins produced by them. These illnesses are present in all types of animals and humans.

Recently with the scare of bio-terrorism there has been an increased concern with pathogens that can produce deadly outbreaks. This is the case with anthrax. Anthrax is considered a potential agent for use in biological warfare. Anthrax is an acute infectious disease caused by the spore-forming bacteria Bacillus anthracis. Anthrax is primarily a disease of domesticated and wild animals, particularly herbivorous animals. Humans become infected with anthrax by handling products from infected animals or by inhaling anthrax spores from contaminated animal products. Anthrax can also be spread by eating undercooked meat from infected animals. Anthrax infection can occur in three forms: cutaneous, inhalation, and gastrointestinal. The most common form is the cutaneous anthrax infection, which occurs when bacteria enter a cut or abrasion on the skin. This infection begins as a raised itchy bump that develops into a vesicle and then a painless ulcer, usually 1-3 cm in diameter, with a characteristic black necrotic area in the center. About 20% of untreated cases of cutaneous anthrax result in death. Deaths may be prevented with prompt antimicrobial treatment. The inhalation form has early symptom similar to a common cold which progressively results in severe breathing problems. This type of anthrax is usually fatal. The intestinal form is characterized by an acute inflammation of the intestinal tract. The initial signs are nausea, loss of appetite, vomiting, and fever followed by abdominal pain, vomiting of blood and severe diarrhea. Intestinal anthrax results in death in 25% to 60% of cases. Anthrax is treated with antimicrobials and can be prevented with vaccination. The Department of Defense in the USA has a mandatory anthrax vaccination of all active military personnel.

Another digestive infection in humans is traveler's diarrhea, which affects over seven million visitors to high-risk tropical and semitropical areas every year. Others suggest that the incidence of traveler's diarrhea is 15-56% among international travelers. Approximately 1% of the sufferers are hospitalized, at least 20% are confined to bed for a day and nearly 40% have to change plans in their travel itinerary.

Traveler's diarrhea, defined as the passage of more than 3 unformed stools in a 24-h period, is a self-limiting illness lasting 3-5 days. The illness may be presented either as (1) acute watery diarrhea (2) diarrhea with blood (dysentery) or (3) chronic diarrhea, often with clinical nutrient malabsorption.

Several factors contribute to the development of diarrhea in travelers, including personal (age, socioeconomic status, body weight, preexisting gastrointestinal illnesses), behavioral (mode of travel, standard of accommodation, eating in public places, dietary errors) and travel related (destination, duration of stay, country of origin, season). Approximately 85% of the diarrheas among international travelers are produced by bacterial enteropathogens. These pathogens are usually acquired through ingestion of fecally contaminated food or water. Sometimes dirty hands or insects are the vectors of fecal contamination. Cooked food is safe to consume as long as the temperature at the interior of the food reaches 160° F. or more. An undercooked hamburger is risky food, because ground meat can become contaminated at the processing plant and during preparation.

The common pathogens that produce traveler's diarrhea include Clostridium difficile, Yersenia enterolitica, Shigella sp., Campylobacter sp., Salmonella sp., ETEC (enterotoxigenic) and EAEC (enteroaggregative) Escherichia coli. Traveler's diarrhea produced by Shigella sp. or Salmonella sp. tend to cause a more severe and longer lasting disease than that caused by the most common cause, enterotoxigenic E. coli (ETEC). Campylobacter jejuni is a relatively common cause of traveler's diarrhea especially in the winter. Viruses such as rotavirus, cytomegalovirus and Norwalk agent are less common causes.

The use of antibiotics limits the course of diarrhea to a little over a day compared with an average of over 3-5 days when diarrhea remains untreated. The widespread resistance of the traditional antimicrobial agent, Trimethoprim plus sulfamethoxazole (TMP/SMX), and fluoroquinolones are the main reasons of concern about the continuous use of antimicrobials for the treatment of traveler's diarrhea (Dupont et al, 1998). The extensive use of antibiotics can also lead to overgrowth syndromes, Candida vaginitis can occur, the overgrowth of Clostridium difficile due to less competitive environment in the gastrointestinal tract can also result in diarrhea.

Short-term travelers that have experience diarrhea do not develop protection, since it requires continued exposure to enteropathogens to develop immunological protection against traveler's diarrhea. Vaccination is a promising option, but vaccines against all enteropathogens that cause traveler's diarrhea have not been developed.

Other protection methods to treat traveler's diarrhea are: the use of nonabsorbed antimicrobials, which have fewer side effects and should be safer to use in children and pregnant women in whom quinolones are contraindicated; antisecretory and antimotility agent (loperamide); the use of attapulgite, a hydrated aluminum silicate clay preparation; and probiotics i.e. lactobacillus, which appear to be useful in the prevention or treatment of travelers diarrhea. In all cases the restoration of water and electrolyte balance is necessary. The following table shows the current treatments for Traveler's Diarrhea:

| Agent | Efficacy | Comments |
|---|---|---|
| Activated charcoal | Not efficacious | May absorb important medications |
| Lactobacillus | Not proven | Safe |
| Bismuth subsalicylate | 65% protective | Rinse mouth to avoid black tongue |
| preparations | | |
| Trimethropim- | 70-80% protective | Resistance rising worldwide |
| sulfamethoxazole | | |
| Fluoroquinolones | 90% protective or | Currently most effective |
| (norfloxacin, ciprofloxacin, | better | antimicrobial but resistance rising |
| ofloxacin) | | worldwide. |

Ericsson, Charles (1998)

In humans and animals, peptic ulcers are open sores produced by a bacteria. These open sores can be present on the entire gastro-intestinal tract, mainly esophagus, stomach and proximal part of the small intestine. There is evidence that support the role of H. pylori as the etiologic agent of chronic gastritis and peptic ulcer. H. pylori, a gram-negative, microaerophilic, spiral bacteria is the major cause of gastro-duodenal disease, including chronic gastritis, gastric and duodenal ulcers and gastric neoplasia. Greater than 50% of North American adults over 50 years of age are infected with H. pylori. In contrast, in some developing and newly industrialized countries virtually all adults are infected. In developing countries almost all children are infected by age 10, whereas in developed countries only the children of lower socioeconomic levels are infected. H. pylori is characterized by very high urease activity that may be associated with virulence, in the absence of urea H. pylori is sensitive to acidic pH. Urease activity may be an important colonization and survival factor by generating ammonia in the immediate bacterial microenvironment. H. pylori has been classified as a type 1 carcinogen by the World Health Organization because of the danger of persistent infection with the bacterium causing gastric cancer. H. pylori infection is of extreme importance in the causation of peptic ulcer disease. By initiating a gastritis or dyspeptic symptoms, it can predispose to subsequent episode of either gastric lymphoma or stomach cancer.

The eradication of H. pylori has been obtained with combination therapy, triple therapy using bismuth plus two antibiotics(metronidazole and either amoxicillin or tetracycline has been effective). Problems due to development of antimicrobial resistant and side effects (diarrhea, nausea, abdominal pain and others) may explain why the use of antibiotics has not become a preferred treatment for gastritis and peptic ulcers due to H. pylori.

Antibacterial treatment of H. pylori is difficult because of the habitat occupied by the organism below the layer of the mucus adherent to the gastric mucosa. Access of antibacterial agents to this site is limited from the lumen of the stomach and also from the gastric blood supply.

The use of medium chain fatty acids and medium chain triglycerides has been shown to inhibit the growth of H. pylori in vitro. The mechanism by which they exert antibacterial effect is thought to involve: 1) damage to the bacterial outer membrane leading the increase membrane fluidity and permeability, 2) Incorporation of these fatty acids, making the bacterial membrane unstable, 3) Production of peroxides due to oxidation of fatty acids. The mode of transmission of H. pylori in humans is still poorly understood. There are reports of detection of this microorganism in the oral cavity and in the feces. If H. pylori is harvested in the oral cavity or bowel, these might represent important reservoir for the reinfection and transmission with consequences from treatment. One vector for the transmission of H. pylori are flies, they can carry viable H. pylori in their external surfaces and alimentary tracts.

In animals, the presence of scours in calves is of economic importance. It is estimated that the death lost of calves less than 6 months of age is approximately 2.5% or over 100,000 a year. Most of the mortality and morbidity of the calves are due to infectious diseases, mainly scours. More than 90% of scours in calves is produced by E. coli and Salmonella. Clostridia has proved to be fatal in the majority of cases. There are preventive methods like (1) vaccination of the mothers in order to passively transfer antibodies in colostrum; (2) the use of immunological supplements for milk replacers; (3) the use of probiotics to create a gastro-intestinal healthy environment (4) changes in calf management. None of these protective measures are 100% effective.

Another animal of economic importance is swine. The incidence of diarrhea in neonates and weaned piglets is very high. Again, E. coli and Salmonella are the main microorganisms involved in diarrhea in swine. There are losses in the nursery while piglets are still lactating and after weaning. There are similar preventive methods as in calves. One of the preferred methods is segregated early weaning (SEW). The basis of early weaning is that the earlier piglets are weaned from the sow the less are the chances of crossover diseases between sow and piglets. This method requires the use of antibiotics.

In both cases, calf and piglet scours, the preferred method of treatment is antibiotics. The European Community has banned the use of 5 antibiotics and in the Unites States the FDA is banning the use of fluoroquinolone in animals due to the development of Campylobacter resistant to this antibiotic. Bacteria resistance has encouraged the development of antibiotic-alternative products.

Terpenes are widespread in nature, mainly in plants as constituents of essential oils. Their building block is the hydrocarbon isoprene (C₅H₈)ₙ. Terpenes have been found to be effective and nontoxic dietary antitumor agents which act through a variety of mechanisms of action (Crowell and Gould, 1994 and Crowell et al, 1996). Terpenes, i.e. geraniol, tocotrienol, perillyl alcohol, b-ionone and d-limonene, suppress hepatic HMG-COA reductase activity, a rate limiting step in cholesterol synthesis, and modestly lower cholesterol levels in animals (Elson and Yu, 1994). D-limonene and geraniol reduced mammary tumors (Elegbede et al, 1984 and 1986 and Karlson et al, 1996) and suppressed the growth of transplanted tumors(Yu et al, 1995).

Terpenes have also been found to inhibit the in-vitro growth of bacteria and fungi (Chaumont and Leger, 1992, Moleyar and Narasimham, 1992 and Pattnaik, et al 1997) and some internal and external parasites (Hooser, et al, 1986). Geraniol was found to inhibit growth of Candida albicans and Saccharomyces cerevisiae strains by enhancing the rate of potassium leakage and disrupting membrane fluidity (Bard, et al, 1988). B-ionone has antifungal activity which was determined by inhibition of spore germination, and growth inhibition in agar (Mikhlin et al, 1983 and Salt et al, 1986). Teprenone (geranylgeranylacetone) has an antibacterial effect on H. pylori (Ishii, 1993). Solutions of 11 different terpenes were effective in inhibiting the growth of pathogenic bacteria in in-vitro tests; levels ranging between 100 ppm and 1000 ppm were effective. The terpenes were diluted in water with 1% polysorbate 20 (Kim et al, 1995). Diterpenes, i.e. trichorabdal A (from R. Trichocarpa) has shown a very strong antibacterial effect against H. pylori (Kadota, et al, 1997).

Rosanol a commercial product with 1% rose oil has been shown to inhibit the growth of several bacteria (Pseudomona, Staphylococus, E. coli and H pylori). Geraniol is the active component (75%) of rose oil. Rose oil and geraniol at a concentration of 2 mg/lt. inhibited the growth of H pylori in vitro. Some extracts from herbal medicines have been shown to have an inhibitory effect on H. pylori, the most effective being decursinol angelate, decursin, magnolol, berberine, cinnamic acid, decursinol and gallic acid (Bae, et al 1998). Extracts from cashew apple, anacardic acid and (E)-2-hexenal, have shown bactericidal effect against H. pylori.

There may be different modes of action of terpenes against H. pylori. They could (1) interfere with the phospholipid bilayer of the cell membrane (2) impair a variety of enzyme systems (HMG-reductase) and (3) destroy or inactivate genetic material.

### SUMMARY OF THE INVENTION

Prevention and treatment of digestive tract infections by orally administering a biocidal terpene, a biocidal terpene mixture or a liposome-terpene(s) composition before of after the onset of the infection.

### DESCRIPTION OF THE PREFERRED EMBODIMENTS

Digestive tract infections not only are an uncomfortable illness for humans but also are of economic importance for the animal industry. In some cases the illness can cause death in children, elderly and immune-compromised people. The preferred treatment of the disease is antibiotics. The extensive use of antibiotics in humans and the animal industry has created the development of antibiotic-resistant bacteria. The increased antibiotic resistance has been the main reason to seek new antimicrobial alternatives. The European Community has banned the use of 5 antibiotics in animals and in the Unites States the FDA is banning the use of fluoroquinolone in animals due to the development of Campylobacter resistant to this antibiotic.

Terpenes, which are GRAS (Generally Recognized As Safe) have been found to inhibit the growth of cancerous cells, decrease tumor size, decrease cholesterol levels and have a biocidal effect on microorganisms in vitro. Onawunmi (1989) showed that growth media with more than 0.01% citral reduced the concentration of E. coli and at 0.08% there was a bactericidal effect. Barranx, et al (1998) teach us a terpene formulation, based on pine oil, used as a disinfectant or antiseptic cleaner. Koga, et al (1998) teach that a terpene found in rice has antifungal activity. Iyer, et al (1999) teach us an oral hygiene antimicrobial product with a combination of 2 or 3 terpenes that showed a synergistic effect. Neither of them suggested the use of a terpene, terpene mixture or liposome-terpene(s) combination for the prevention or treatment of gastro-intestinal infections i.e. traveler's diarrhea.

Several U.S. patents (U.S. Pat. Nos. 5,547,677, 5,549,901, 5,618,840, 5,629,021, 5,662,957, 5,700,679, 5,730,989) teach us that certain types of oil-in-water emulsions have antimicrobial, adjuvant and delivery properties.

Liposomes are microscopic structures consisting of concentric lipid bilayers enclosing an aqueous space. Liposomes are classically prepared from phospholipids which occur naturally in animal cell membranes, but several synthetic formulations are now commonly used. The lipid composition of the liposome can be varied to give liposomes different physical characteristics i.e. size and stability. Liposomes can be prepared by the reverse-phase evaporation or dehydration-rehydration vesicle methods using a mixture of dipalmitoyl phosphatidyl choline, cholesterol, dipalmitoyl phosphatidyl glycerol, dipalmitoyl phosphatidyl ethanolamine and other synthetic fatty acids and emulsifiers. When making liposomes first multilamellar vesicles are formed spontaneously when amphipathic lipids are hydrated in an aqueous medium. Unilamellar vesicles are often produced from multilamellar vesicles by the application of ultrasonic waves.

Multilamellar vesicles can be prepared by the procedure known as dehydration-rehydration. Briefly, egg phosphatidylcholine and cholesterol are mixed in chloroform, dried in a rotary evaporator, dilute with water and sonificated to form unilamellar vesicles. The solution is freeze dried and rehydrated with the terpene solution in order to embed the terpene inside the liposome. Another method to produce liposomes is by mixing together lipids, an emulsifier and the terpenes. The emulsion is obtained by using a Polytron homogenizer with special flat rotor that creates an emulsion. The lipids could consist of soybean oil, any commercial or pharmaceutical oil; the emulsifier consist of egg yolk lecithin, plant sterols or synthetic including polysorbate-80, polysorbate-20, polysorbate-40, polysorbate-60, polyglyceryl esters, polyglyceryl monooleate, decaglyceryl monocaprylate, propylene glycol dicaprilate and triglycerol monostearate. The lipid concentration in the oil phase is 75-95% and the emulsifier concentration from 5-25%. When preparing the emulsion a ratio oil to water could vary from 10-15 parts lipid to 35-40 parts terpenes diluted in water at a concentration of 0.5% to 50%, Once the emulsion is formed this is combined with a carrier in order to be use as a humectant, cream or other suitable carrier for topical applications. The emulsion concentration use for topical application varies from 0.0055 through 1.0% of the final product. Several modifications to the emulsion can be achieved by simply varying the concentration and type of terpenes used. This modifications can give us different products with different antimicrobial specificity.

By encapsulating terpenes within these emulsions the antimicrobial effect will be increased: (1) the liposome will disrupt the bacterial membrane and (2) the terpenes will be more effective in disrupting cytoplasmatic enzymes.

It will be apparent for those skilled in the art that the aforementioned objects and other advantages may be further achieved by the practice of the present invention.

### EXAMPLE 1

### Preparation of the Terpene Mixture

The terpene, terpene mixture or liposome-terpene(s) combination consists of a blend of generally recognized as safe (GRAS) terpenes with a GRAS surfactant. The ratio of terpenes is from 1-99% and the surfactant ratio from 1-99% of the mixture. The terpenes, comprised of natural or synthetic terpenes, are citral, b-ionone, geraniol, eugenol, carvone, terpeniol, carvacrol, anethole or other terpenes with similar properties. The surfactant is preferably polysorbate-80 or other suitable GRAS surfactants.

### EXAMPLE 2

### Preparation of Liposomes Containing Terpenes

Any standard method for the preparation of liposomes can be followed with the knowledge that the lipids used are all food-grade or pharmaceutical-grade. A set amount of lipids, an emulsifier and the terpenes was used to prepare an emulsion. The emulsion was obtained by using a Polytron homogenizer with special flat rotor that created an emulsion. The lipids consisted of soybean oil, any commercial or pharmaceutical oil; the emulsifier consist of egg yolk lecithin, plant sterols or synthetic including polysorbate-80, polysorbate-20, polysorbate-40, polysorbate-60, polyglyceryl esters, polyglyceryl monooleate, decaglyceryl monocaprylate, propylene glycol dicaprilate and triglycerol monostearate. A solution containing 75-95% lipids (oil) and 5-25% emulsifier consisted of the oil phase. The aqueous phase consisted of the terpene diluted in water at a rate of 0.5% to 50%. To form the emulsion a ratio oil to water varying from 10-15 parts lipid (oil phase) to 35-40 parts terpenes (aqueous phase) was mixed. Any standard method for the preparation of liposomes can be followed with the knowledge that the lipids used are all food-grade or pharmaceutical-grade. The suspension containing a lipid, an emulsifier and the terpenes is emulsified with a Posytron homogenizer until a complete milky solution is obtained.

### EXAMPLE 3

### Preparation of Liposome

This step consists of the preparation of the terpene(s)-liposome combination by mixing 99% of liposome and 1% of terpene mixture. Several combinations of this formulation can be obtained by varying the amount of terpene and liposome from 1% to 99%. The liposomes are prepared as in Example 1 without the addition of terpenes in the formulation.

### EXAMPLE 4

### In-vitro Effectiveness of Terpenes Against E. coli

This example demonstrates the effect of terpenes on the cell membrane fragility of E. coli, which is considered indicative of other pathogenic bacteria such as Salmonella and Listeria. Lysis of the cell membrane was monitored by the determination of galactosidase activity. B-galactosidase is a well-characterized cytosolic enzyme in bacteria. This enzyme is inducible in the presence of isopropyl-1-thiogalactosidase (IPTG) and assayed colorimetricaly with substrate o-nitro-phenyl-B-D-galactoside (ONPG). ONPG is cleaved to release o-nitrophenol with peak absorbance at 420 nm. Since intact E. coli is impermeable to both ONPG and the enzyme, the cells have to be lysed prior to enzymatic assay. Therefore the ability of terpenes to lyse E. coli can be measured with this enzymatic assay and compared to known lysing agents. The procedure used was as follows: E. coli strains AW574 or AW405 were cultured overnight in 10 ml tryptone broth with 1 nM IPTG at 35° C. Cells were allowed to growth after an absorbance equal to 0.9 was reached. Cells were harvested, washed with phosphate buffer and resuspended to an absorbance equal to 0.5. One tenth of a ml of the bacteria culture was added to 0.9 ml of buffer, warmed to 30° C. and then 80 ul of terpenes (85% terpenes and 15% polysorbate-80), 80 ul water (background) or 40 ul chloroform plus 40 ul 1% SDS in water (positive control) were added. After the addition of the lysing agents the tubes were mixed for 10 seconds and 0.2 ml of ONPG (4 mg/ml water) was added, then incubated for 5 minutes. The enzyme activity was stopped with 0.5 ml of 1 M sodium carbonate. After being centrifuged for 3 minutes at 1,500×g, supernatant was transferred to cuvettes and read at 420 nm. The relative degree of lysis caused by terpenes was calculated as follows:

100×(OD terpenes−OD water)/(OD chloroform−OD water)

This shows that dosages can be manipulated to either lyse the cell outright, or in the case of lower dosages, stop bacterial growth without lysis of the cell membrane. The advantage of this controllable result is the ability to prevent lysis and the resultant release of endotoxins where contraindicated.

TABLE 1
Lysis of E. coli by Terpenes

| Terpenes | | | Relative lysis |
|---|---|---|---|
| (uM) | | | % |
| | Carvone | 404,000 | NM* |
| | | 40,400 | 54 |
| | | 4,040 | 22 |
| | | 404 | 3.2 |
| | Geraniol | 363,000 | NM |
| | | 36,300 | 96 |
| | | 3,630 | 98 |
| | | 363 | 34 |
| | | 36.3 | 4 |
| | | 3.63 | 2.4 |
| | b-Ionone | 308,000 | NM |
| | | 30,800 | NM |
| | | 3,080 | NM |
| | | 308 | 52 |
| | | 30.8 | 44 |
| | | 3.08 | 23 |
| | | 0.308 | 4.78 |
| | | 0.0308 | 1.3 |

| | |
|---|---|
| 80 ul Polysorbate-80 | 3.2 |
| 80 ul Polysorbate-80 + SDS + Chloroform | 100 |
| SDS + Chloroform | 100* |

*Lysis due to chloroform and SDS combination was considered to be 100%.
*NM not measurable due to formation of turbid colloidal solution.

### EXAMPLE 5

### In In-vitro Effectiveness of Terpenes Against Several Microorganisms

This example demonstrate the effectiveness of the terpene mixture against Escherichia coli, Salmonella typhimurium, Pasteurella mirabilis, Staphylococcus aureus, Candida albicans and Aspergillius fumigatus. Each organism, except A. fumigatus, was grown overnight at 35-37° C. in tryptose broth. A. fumigatus was grown for 48 hours. Each organism was adjusted to approximately 10⁵ organisms/ml with sterile saline. For the broth dilution test, terpene mixture was diluted in sterile tryptose broth to give the following dilutions: 1:500, 1:1000, 1:2000, 1:4000, 1:8000, 1:16,000, 1:32,000, 1:64,000 and 1:128,000.

Each dilution was added to sterile tubes in 5 ml amounts. Three replicates of each series of dilutions were used for each test organism. One half ml of the test organism was added to each series and incubated at 35-37° C. for 18-24 hours. After incubation the tubes were observed for growth and plated onto blood agar. The tubes were incubated an additional 24 hours and observed again. The A. fumigatus test series was incubated for 72 hours. The minimum inhibitory concentration for each test organism was determined as the highest dilution that completely inhibits the organism.

TABLE 2
Results of the inhibitory activity of different dilutions

| | Visual assessment of | | | Growth after subculture to | | | Mean |
|---|---|---|---|---|---|---|---|
| | growth * | | | agar plates * | | | inhibitory |
| Organism | 1 | 2 | 3 | 1 | 2 | 3 | dilution |
| S. typhimurium | 500 | 500 | 500 | 500 | 500 | 500 | 500 |
| E. coli | 1000 | 1000 | 1000 | 1000 | 1000 | 1000 | 1000 |
| P. mirabilis | 1000 | 1000 | 1000 | 1000 | 1000 | 1000 | 1000 |
| S. aureus | 1000 | 1000 | 1000 | 1000 | 1000 | 1000 | 1000 |
| C. albicans | 1000 | 1000 | 1000 | 1000 | 1000 | 1000 | 1000 |
| A. fumigatus | 8000 | 16,000 | 16,000 | 8000 | 16,000 | 16,000 | 13,300 |

* The results of the triplicate tests with each organism as the reciprocal of the dilution that showed inhibition/killing
** NI = not inhibited

### EXAMPLE 6

### In In-vitro Effectiveness of Terpenes Against Escherichia coli Over Time

This example demonstrates the effectiveness of the terpene mixture at several concentrations against Escherichia coli and cultured over time. Terpene dilutions (1:500, 1:1000, 1:2000, 1:4000, 1:8000, and 1:16,000) were prepared in BHI broth and in saline. These were prepared in 25 ml amounts. E. coli was grown overnight in BHI broth and diluted to a MacFarland 0.5 concentration in saline. This solution was diluted 1:100 to be used to inoculate (0.5 ml) each terpene dilution tube. The series that contained the terpene dilution in BHI was tested at 30 min, 90 min, 150 min and 450 min. Each tube was mixed and serially diluted in saline. One half milliliters of each dilution was spread plated onto MacConkey (MAC) agar plates. Also, 3 drops of the undiluted and the 1:100 dilution was added into respective tubes of BHI broth. The tubes and plates were incubated overnight at 35° C. The series that contained the terpene's dilution in saline were tested at 60 min, 120 min, 180 min and 480 min. Each tube was mixed and serially diluted in saline. One half milliliters of each dilution was spread plated onto MacConkey (MAC) agar plates. Also, 3 drops of the undiluted and the 1:100 dilution were added into respective tubes of BHI broth. The tubes and plates were incubated overnight at 35° C.

TABLE 3
Subculture from the tubes containing various dilutions of terpenes in broth

| Time | Dilution | 1:500 | 1:1000 | 1:2000 | 1:4000 | 1:8000 | 1:16,000 |
|---|---|---|---|---|---|---|---|
| 30 min | Undiluted | NG | + | + | + | + | + |
| | 1:100 | NG | + | + | + | + | + |
| 90 min | Undiluted | NG | NG | + | + | + | + |
| | 1:100 | NG | NG | NG | + | + | + |
| 150 min | Undiluted | NG | NG | + | + | + | + |
| | 1:100 | NG | NG | NG | + | + | + |
| 450 min | Undiluted | NG | NG | + | + | + | + |
| | 1:100 | NG | NG | + | + | + | + |

NG: no growth,
+ growth

TABLE 4
Subculture from the tubes containing various dilutions of terpenes in saline

| Time | Dilution | 1:500 | 1:1000 | 1:2000 | 1:4000 | 1:8000 | Control |
|---|---|---|---|---|---|---|---|
| 60 min | Undiluted | NG | + | + | + | + | + |
| | 1:100 | NG | NG | NG | + | + | + |
| 120 min | Undiluted | NG | NG | NG | + | + | + |
| | 1:100 | NG | NG | NG | NG | + | + |
| 180 min | Undiluted | NG | NG | NG | + | + | + |
| | 1:100 | NG | NG | NG | NG | + | + |
| 480 min | Undiluted | NG | NG | NG | NG | + | + |
| | 1:100 | NG | NG | NG | NG | NG | + |

NG: no growth,
+ growth

TABLE 5
The quantitative results of the activity of various terpene dilutions against E. coli
(cfu)

| Media | Time | 1:500 | 1:1000 | 1:2000 | 1:4000 | 1:8000 | Control |
|---|---|---|---|---|---|---|---|
| Broth | 30 min | 0 | 0 | 660 | 3600 | 3600 | 4600 |
| | 90 min | 0 | 0 | 12 | 4600 | 5400 | 7600 |
| | 150 min | 0 | 0 | 10 | 8000 | 12,000 | 14,000 |
| | 450 min | 0 | 0 | 15,000 | 28 × 10³ | 23 × 10⁷ | 16 ×10⁸ |
| Saline | 60 min | 0 | 4 | 140 | 4000 | 2000 | 1300 |
| | 120 min | 0 | 0 | 0 | 90 | 3800 | 2600 |
| | 180 min | 0 | 0 | 0 | 2 | 2000 | 5000 |
| | 480 min | 0 | 0 | 0 | 0 | 104 | 8000 |

NG: no growth,
+ growth

### EXAMPLE 7

### In vitro Effectiveness of Selected Terpenes on Helicobacter pylori

This example shows the bactericidal effect of selected terpenes on the viability of H. pylori. Five terpenes (anethole, carvone, citral, geraniol and b-ionone) were used for this study. Terpenes were mixed to a ratio of 90% terpene plus 10% polysorbate-80. The H. pylori used was strain #26695 of porcine origin, this bacteria is a motile, cag A, vac A cytotoxin-positive gram negative bacteria which colonizes gnotobiotic piglets and indefinitely persists within the gastric microenvironment as a superficial infection of the gastric mucosa and mucus layer.

The study was as follows:

1) Stock solutions of each terpene with polysorbate-80 were prepared (1.8 ml terpene plus 0.2 ml polysorbate-80.

2) Stock solutions were diluted in Brucella broth 10% (v/v) fetal calf serum to a final concentration of stock at 1:10, 1:50, 1:100, 1:500, 1:1000, 1:5000 and 1:10000. Controls consisted of 10% (v/v) polysorbate-80 in Brucella broth, Brucella broth alone and bacteria in Brucella broth.

3) A total of 1.0×10⁶ bacteria (30 ul) was added to 970 ul terpene dilutions (final volume of 1.0 ml) in loosely capped tubes and incubated for 24 hours at 37° C. with continuous mixing.

4) Duplicate samples (0.1 ml) from each test dilution was titrated onto blood agar plates and incubated for 48 hours at 37° C. on 10% CO₂ environment. Bacterial colony forming units (cfu) were determined visual (counting) inspection. Recovered bacteria were confirmed to be H. pylori by catalase and urease enzyme activities.

The results are summarized in the following table:

TABLE 6
Effect of different terpenes on H. pylori growth

| | Final dilution tested for antimicrobial effects against 10⁶ cfu | | | | | | | |
|---|---|---|---|---|---|---|---|---|
| Terpene | 1:10 | 1:50 | 1:100 | 1:500 | 1:1000 | 1:5000 | 1:10000 | 1:50000 |
| Polysorbate-80 | NG* | NG | NG | 10³ | 10⁴ | TNTC** | TNTC | TNTC |
| Anethole | NG | NG | NG | NG | 10³ | 10³ | TNTC | TNTC |
| Carvone | NG | NG | NG | NG | NG | 10⁴ | TNTC | TNTC |
| Geraniol | NG | NG | NG | NG | NG | NG | 10² | TNTC |
| Citral | NG | NG | NG | NG | NG | NG | NG | TNTC |
| b-ionone | NG | NG | NG | NG | NG | NG | NG | TNTC |

*NG = no growth
**TNTC = Too Numerous To Count

### EXAMPLE 8

### In vitro Effectiveness of Single or Combination of Terpenes Against E. coli

The objective of this example was to determine an optimum terpene mixture which could have a greater biocidal effect. E. coli strain AW574 was grown in tryptone broth to an exponential growth phase (O.D. between 0.4 and 1.0 at 590 nm). One tenth of this growth was inoculated to 10 ml of tryptone broth following by the addition of individual terpenes or as indicated on table 2; then incubated for 24 hours at 35-37° C. and the O.D. determined in each tube. The concentration of terpenes was 1 or 2 uMol. Each treatment was repeated in triplicate. The results are expressed as percentage bacterial growth as compared to the control treatment. It is observed that the combination of terpenes give better biocidal effect than single terpenes, with geraniol and carvone better than b-ionone.

TABLE 7
Effect of single terpene or their combination
against on E. coli growth

| uMol terpenes | | | % |
|---|---|---|---|
| B-ionone | Carvone | Geraniol | growth |
| 0 | 0 | 0 | 100.00 |
| 2 | 0 | 0 | 84.00 |
| 0 | 2 | 0 | 63.00 |
| 0 | 0 | 2 | 54.00 |
| 1 | 1 | 1 | 41.00 |
| 1 | 2 | 1 | 31.10 |
| 1 | 1 | 2 | 14.80 |
| 1 | 2 | 2 | 15.90 |
| 2 | 1 | 1 | 48.60 |
| 2 | 2 | 1 | 44.30 |
| 2 | 1 | 2 | 30.20 |
| 2 | 2 | 2 | 1.50 |

It will be apparent for those skilled in the art that a number of modifications and variations may be made without departing for the scope of the present invention as set forth in the appending claims.

### REFERENCES

1. Bae E A, M J Han, N J Kim and D H Kim, 1998. Anti-Helicobacter pylori activity of herbal medicines. Biol, Pharm. Bull 21(9) 990-992.

2. Bard, M, M R Albert, N Gupta, C J Guuynn and W Stillwell, 1988. Geraniol interferes with membrane functions in strains of Candida and Saccharomyces. Lipids 23(6): 534-538.

3. Barranx A, M Barsacq, G Dufau and J P Lauilhe, 1998. Disinfectant or antiseptic composition comprising at least one terpene alcohol and at least one bactericidal acidic surfactant, and use of such a mixture. U.S. Pat. No. 5,763,468.

4. Boyanova L and G Neshev, 1999. Inhibitory effect of rose oil products on Helicobacter pylori growth in vivo: preliminary report. J. Med. Microbiol. 48: 705-706.

5. Chaumont J P and D Leger, 1992. Campaign against allergic moulds in dwellings. Inhibitor properties of essential oil geranium “Bourbon”, citronellol, geraniol and citral. Ann Pharm Fr 50(3): 156-166.

6. Crowell, P L and M N Gould, 1994. Chemoprevention and therapy of cancer by d-limonene. Crit Rev Oncog 5(1): 1-22.

7. Crowell, P L, S Ayoubi and Y D Burke, 1996. Antitumorigenic effects of limonene and perillyl alcohol against pancreatic and breast cancer. Adv Exp Med Biol 401: 131-136.

8. Dupont H. L., C. D. Ericsson, J. J. Mathewson, M. W. Dupont, Z. D. Jiang, A. Mosavi and F. J. de la Cabaña, 1998. Rifaximin: a nonabsorved antimicrobial in the therapy of traveler's diarrhea. Digestion 59: 708-714.

9. Elegbede, J A, C E Elson, A Qureshi, M A Tanner and M N Gould, 1984. Inhibition of DMBA-induced mammary cancer by monoterpene d-limonene. Carcinogenesis 5(5): 661-664.

10. Elegbede, J A, C E Elson, A Qureshi, M A Tanner and M N Gould, 1986. Regression of rat primary mammary tumors following dietary d-limonene. J Natl Cancer Inst. 76(2): 323-325.

11. Elson, C E and S G Yu, 1994. The chemoprevention of cancer by mevalonate-derived constituents of fruits and vegetables. J Nutr. 124: 607-614.

12. Ericsson, Charles, 1998. Traveler's diarrhea: Epidemiology, prevention and self-treatment. Travel Medicine 12 (2): 285-303.

13. Grubel P and D R Cave, 1998. Sanitation and houseflies (musca domestica):factors for the transmission of Helicobacter pylori. Bull. Inst. Pasteur 96: 83-81.

14. Hooser, S B, V R Beasly and J J Everitt, 1986. Effects of an insecticidal dip containing d-limonene in the cat. J Am Vet Med Assoc. 189(8): 905-908.

15. Ishii, E., 1993. Antibacterial activity of teprenone, a non water-soluble antiulcer agent, against Helicobacter pylori. Int. J Med Microbiol Virol. Parasitol. Infect Dis. 280(1-2): 239-243.

16. Iyer L M, J R Scott, and D F Whitfield, 1999. Antimicrobial compositions. U.S. Pat. No. 5,939,050.

17. Kadota S, P Basnet, E Ishii, T Tamura and T Namba, 1997. Antibacterial activity of trichorabdal A from Rabdosia trichocarpa against Helicobacter pylori. Zentralbl. Bakteriol 287(1): 63-67.

18. Karlson, J, A K Borg, R Unelius, M C Shoshan, N Wilking, U Ringborg and S Linder, 1996. Inhibition of tumor cell growth by monoterpenes in vitro: evidence of a Ras-independent mechanism of action. Anticancer Drugs 7(4): 422-429.

19. Kim J, M Marshall and C Wei, 1995. Antibacterial activity of some essential oil components against five foodborne pathogens. J Agric Food Chem. 43: 2839-2845.

20. Koga, J, T Yamauchi, M Shimura, Y Ogasawara, N Ogasawara and J Suzuki, 1998. Antifungal terpene compounds and process for producing the same. U.S. Pat. No. 5,849,956.

21. Kubo J, J R Lee and I Kubo, 1999. Anti-Helicobacter pylori agents from the cashew apple. J Agric Food Chem. 47: 533-537.

22. Iyer L M, J R Scott, and D F Whitfield, 1999. Antimicrobial compositions. U.S. Pat. No. 5,939,050.

23. Mikhlin E D, V P Radina, A A Dmitrossky. L P Blinkova and L G Button, 1983. Antifungal and antimicrobial activity of some derivatives of beta-ionone and vitamin A. Prikl Biokhim Mikrobiol. 19: 795-803.

24. Moleyar V and P Narasimham, 1992. Antibacterial activity of essential oil components. Int J Food Microbiol 16(4): 337-342.

25. Onawunmi, G O, 1989. Evaluation of the antimicrobial activity of citral. Letters in Applied Microbiology 9(3): 105-108.

26. Pattnaik, S, V R Subramanyan, M Bapaji and C R Kole, 1997. Antibacterial and antifungal activity of aromatic constituents of essential oils. Microbios 89(358): 39-46.

27. Petschow B W, R P Batema and L L Ford, 1996. Susceptibility of Helicobacter pylori to bactericidal properties of medium-chain monoglycerides and free fatty acids. Antimicrobial Agents and Chemotherapy 40 (2): 302-306.

28. Salt, S D, S Tuzun and J Kuc, 1986. Effects of B-ionone and abscisic acid on the growth of tobacco and resistance to blue mold. Mimicry the effects of stem infection by Peronospora tabacina. Adam Physiol Molec Plant Path 28: 287-297.

29. Wright, D C, 1996. Antimicrobial oil-in-water emulsions. U.S. Pat. No. 5,547,677.

30. Wright, D C, 1996. Antimicrobial oil-in-water emulsions. U.S. Pat. No. 5,549,901.

31. Wright, D C, 1997. Antimicrobial oil-in-water emulsions. U.S. Pat. No. 5,618,840.

32. Wright, D C, 1997. Micellar nanoparticles. U.S. Pat. No. 5,629,021.

33. Wright, D C, 1997. Oil containing lipid vesicles with marine applications. U.S. Pat. No. 5,662,957.

34. Wright, D C, 1997. Lipid vesicles having a bilayer containing a surfactant with anti-viral and spermicidal activity. U.S. Pat. No. 5,700,679.

35. Wright, D C, 1998. Oral vaccine against gram negative bacterial infection. U.S. Pat. No. 5,730,989.

36. Yu, S G, P J Anderson and C E Elson, 1995. The efficacy of B-ionone in the chemoprevention of rat mammary carcinogenesis. J Agri Food Chem 43: 2144-2147.

What is claimed is:

1. A method to prevent or treat digestive tract infections by orally ingesting a solution or composite comprising a terpene, a mixture of terpenes or a liposome-terpene(s) combination with or without a surfactant, before or after the onset of said digestive infection; and said method comprising biocidal terpenes whose biocidal properties synergistically increase when two or more terpenes are present in the same composition.

2. A method of claim 1, wherein the terpene, terpene mixture or liposome-terpene(s) combination consist of 1 to 99% terpenes and 1 to 99% surfactant.

3. A method of claim 1, wherein the terpene or terpene mixture are natural or synthetic terpenes selected from citral, b-ionone, geraniol, carvacrol, eugenol, carvone, terpeniol, anethole or other generally recognized as safe terpenes with biocidal properties, and the surfactant is selected from polysorbate-80, polysorbate-20, polysorbate-40, polysorbate-60, polyglyceryl esters, polyglyceryl monooleate, decaglyceryl monocaprylate, propylene glycol dicaprilate, triglycerol monostearate or their combination.

4. A method of claim 1, wherein the terpene does not facilitate microorganism mutation in order for said microorganism to become resistant to the terpene.

5. A method of claim 1, wherein the liposome-terpene(s) combination comprises the encapsulation of the terpene or terpene mixture within the liposome.

6. A method of claim 1, wherein the liposome-terpene(s) combination comprises mixing liposomes with the terpene or the terpene mixture.

7. A method of claim 1, wherein the terpene, terpene mixture or the liposome-terpene(s) combination is part of the inner core of a gelatin or cellulose capsule.

8. A method of claim 1, wherein the terpene, terpene mixture or the liposome-terpene(s) combination is freeze dried, spray dried or dried in order to form a powder for encapsulation or solubilization.

9. A method of claim 1, wherein the terpene, terpene mixture or the liposome-terpene(s) combination is freeze dried, spray dried or dried in order to be compressed in pill or tablet form.

10. A method of claim 1, wherein the terpene, terpene mixture or the liposome-terpene(s) combination is freeze dried, spray dried or dried in order to be compressed in pill/tablet and coated for absorption in different areas along the gastro-intestinal tract.

11. A method of claim 1, wherein the terpene, terpene mixture or the liposome-terpene(s) combination is used for the treatment or prevention of digestive tract infections in humans and animals.

12. A method of claim 1, wherein the digestive tract infections in humans and animals are produced by normal, pathogenic or opportunistic microorganisms or its toxins selected from Aerobacter sp., Aspergillus sp., Bacillus sp., Campylobacter sp., Candida sp., Clostridia sp., Enterobacteriaceae sp., Enterococcus sp., Escherichia sp., Haemophilus sp., Helicobacter sp Klebsiella sp., Lactobacillus sp., Listeria sp., Propionibacter sp., Pasteurella sp., Proteus sp., Pseudomonas sp., Salmonella sp., Shigella sp., Staphylococcus sp., Streptococcus sp. and Yersennia sp.

13. A method of claim 1, wherein the terpene, terpene mixture or the liposome-terpene(s) combination is effective against pathogenic and normal microflora comprising of Aerobacter sp., Aspergillus sp., Bacillus sp., Campylobacter sp., Candida sp., Clostridia sp., Enterobacteriaceae sp., Enterococcus sp., Escherichia sp., Haemophilus sp., Helicobacter sp Klebsiella sp., Lactobacillus sp., Listeria sp., Propionibacter sp., Pasteurella sp., Proteus sp., Pseudomonas sp., Salmonella sp., Shigella sp., Staphylococcus sp., Streptococcus sp. and Yersennia sp.

14. A method of claim 1, wherein the terpene, terpene mixture or the liposome-terpene(s) combination is effective against pathogenic and opportunistic microorganisms causing traveler's diarrhea.

15. A method of claim 1, wherein the terpene, terpene mixture or the liposome-terpene(s) combination is effective against pathogenic and opportunistic microorganisms causing ulcers along the digestive tract.

16. A method of claim 1, wherein the terpene, terpene mixture or the liposome-terpene(s) combination is effective against anthrax.

17. A method of claim 1, wherein the terpene, terpene mixture or the liposome-terpene(s) combination is effective against pathogenic and opportunistic microorganisms causing scours in calves.

18. A method of claim 1, wherein the terpene, terpene mixture or the liposome-terpene(s) combination is effective against pathogenic and opportunistic microorganisms causing scours in neonates and weaned piglets.

19. A method of claim 1, wherein the terpene, terpene mixture or the liposome-terpene(s) combination at lower concentrations has a bacteriostatic effect against pathogenic and normal gastro-intestinal microflora.

20. A method of claim 1, wherein the terpene, terpene mixture or the liposome-terpene(s) combination at higher concentrations has a bactericidal effect against pathogenic and normal gastro-intestinal microflora.

21. A method of claim 1, wherein the effective dose of the terpene, the mixture of terpenes or the liposome-terpene(s) combination is between 20 mg and 5000 mg.

22. A method in claim 1, wherein the effective dose of the terpene, the mixture of terpenes or the liposome-terpene(s) combination is between 20 ppm and 50000 ppm in water and/or food consumed by the human or animal.

23. A method in claim 1, wherein the terpene, the mixture of terpenes or the liposome-terpene(s) combination is prepackaged in liquid form for oral consumption by humans or animals.

24. A method in claim 1, wherein the terpene, a mixture of terpenes or the liposome-terpene(s) combination is mixed with milk replacer and fed to calves and piglets.

25. A method in claim 1, wherein the terpene, the mixture of terpenes or the liposome-terpene(s) combination is intubated directly into the stomach of an animal.
